(19) United States
(12) Patent Application Publication
(10) Pub. No.: US 2012/0041531 A1
(43) Pub. Date: Feb. 16, 2012

(54) COCHLEAR IMPLANT WITH IMPROVED LUMEN ARRANGEMENT

(75) Inventors: Fysh Dadd, New South Wales (AU); Edmond D. Capcelea, New South Wales (AU); Claudiu Treaba, Centennial, CO (US)

(73) Assignee: COCHLEAR LIMITED, New South Wales (AU)

(21) Appl. No.: 12/745,691

(22) PCT Filed: Dec. 1, 2008
(86) PCT No.: PCT/AU08/01776

§ 371 Date: Aug. 24, 2010

(30) Foreign Application Priority Data
Nov. 30, 2007 (AU) 2007906554

(51) Int. Cl. A61F 11/04; A61N 1/05

(52) U.S. Cl. 607/137; 607/116

(57) ABSTRACT

Disclosed is an electrode lead for a medical implant, and in one example, a cochlear implant. The electrode lead includes a lumen for receiving a stylet for assisting in implanting the device. The lumen of the electrode lead is removable from the electrode lead upon or after implantation.

### CROSS-REFERENCE TO RELATED APPLICATION

The present invention is a National Stage Application of International Application No. PCT/AU2008/001776, filed Dec. 1, 2008, which claims priority from Australian Patent Application No. 2007906554, filed Nov. 30, 2007, which is hereby incorporated herein by reference herein.

### BACKGROUND

1. Field of Invention

The present invention relates generally to cochlear implants having a lumen for receiving a stylet, and more particularly, to a cochlear implant with an improved lumen arrangement.

2. Related Art

A cochlear implant allows for electrical stimulating signals to be applied directly to the auditory nerve fibres of the patient, allowing the brain to perceive a hearing sensation approximating the natural hearing sensation. These stimulating signals are applied by an array of electrodes implanted into the patient's cochlea.

The electrode array is connected to a stimulator unit which generates the electrical signals for delivery to the electrode array. The stimulator unit in turn is operationally connected to a signal processing unit which also contains a microphone for receiving audio signals from the environment, and for processing these signals to generate control signals for the stimulator.

When inserting the implant into the patient's cochlea, great care must be taken to avoid damaging the delicate structure of the cochlea. Since the cochlea is a coiled structure, many cochlear implants are manufactured to have a natural curve to facilitate insertion and to fit more naturally in the cochlea once implanted.

However, while inserting a cochlear implant into the cochlea, it is necessary to straighten portions of the implant as it is being inserted, and then to allow it to naturally reassume its curled state after insertion by extracting the stylet from the lumen. To provide a means for straightening the implant, a rigid or semirigid spike, known as a stylet is used as a “spine” for the implant as it is being inserted. A portion of the implant, known as a lumen, receives the stylet during the insertion process. FIG. 1A shows a side view of a cochlear implant electrode lead 10, for supporting an array of electrode contacts (not shown) with lumen 30 and stylet 20. Stylet 20 is shown inserted into lumen 30 to straighten the natural curve of the electrode lead 10.

FIG. 1B is a cross-sectional view of the electrode 10 in FIG. 1 along the line A-A′. FIG. 1B shows electrode lead 10 with lumen 30 and stylet 20 inserted in the lumen 30. FIG. 1B also shows electrode conductive wires 11 and electrode contact 12, associated with one of the electrode conductive wires. The plurality of electrode conductive wires 11 and respective electrode contacts 12 form the electrode array supported by electrode lead 10.

### SUMMARY

According to one aspect of the present invention, an electrode lead for a medical implant is disclosed. The electrode lead comprises: a lumen portion at least partially defining a lumen for receiving a stylet; and a main portion configured to carry one or more electrodes, wherein the main portion is removably connected to the lumen portion.

In accordance with another aspect of the preset invention, a cochlear implant is disclosed. The cochlear implant comprises a stimulator and an electrode lead supporting an array of electrode contacts, the electrode lead comprising: a lumen portion at least partially defining a lumen for receiving a stylet; and a main portion configured to carry one or more electrodes, wherein the main portion is removably connected to the lumen portion.

### BRIEF DESCRIPTION OF THE DRAWINGS

Various aspects and embodiments of the present invention will now be described in detail with reference to the following drawings in which:

FIG. 1A shows a prior art electrode lead for a cochlear implant;

FIG. 1B is a cross-sectional view of the electrode lead of FIG. 1A;

FIG. 2 is a cross-sectional view of an electrode lead for a cochlear implant according to one embodiment of a first aspect of the present invention;

FIG. 3A is a cross-sectional view of another embodiment of an electrode lead for a cochlear implant according to the first aspect of the present invention;

FIG. 3B shows the arrangement of FIG. 3A after separation;

FIG. 4A is a cross-sectional view of an electrode lead for a cochlear implant according to another embodiment of the first aspect of the present invention;

FIG. 4B is a cross-sectional side view of the electrode lead of FIG. 4A;

FIG. 5 is a cross-sectional view of a further embodiment of the electrode lead for a cochlear implant according to the first aspect of the invention;

FIG. 6 is a perspective view of a further embodiment of the electrode lead according to the first aspect of the present invention;

FIG. 7 is a side view of one embodiment of an electrode lead for a cochlear implant according to a second and third aspect of the present invention;

FIG. 8 is a side view of another embodiment of an electrode lead for a cochlear implant according to the second and third aspects of the present invention;

FIG. 9 is a side view of a further embodiment of an electrode lead for a cochlear implant according to the second and third aspects of the present invention;

FIG. 10 is a side view of yet a further embodiment of an electrode lead for a cochlear implant according to both the first and the second aspects of the present invention combined;

FIG. 11 is a side view of yet a further embodiment of an electrode lead for a cochlear implant according to both the first and the second aspects of the present invention combined;

FIG. 12A is a cross-sectional view of one embodiment of an electrode lead for a cochlear implant according to a fourth aspect of the present invention;

FIG. 12B shows the electrode lead of FIG. 12A showing swelling of the swelling polymer; and

FIG. 12C shows the electrode lead of FIGS. 12A and 12B showing further swelling of the swelling polymer;

FIG. 12D shows the electrode lead of FIGS. 12A, 12B and 12C showing even further swelling;

FIG. 13A shows an electrode lead according to another embodiment of the present invention;

FIG. 13B shows the electrode lead of FIG. 13 A with the lumen removed; and

FIG. 14 shows an example of a cochlear implant with an electrode lead to which one or more of the various aspects of the present invention may be applied.

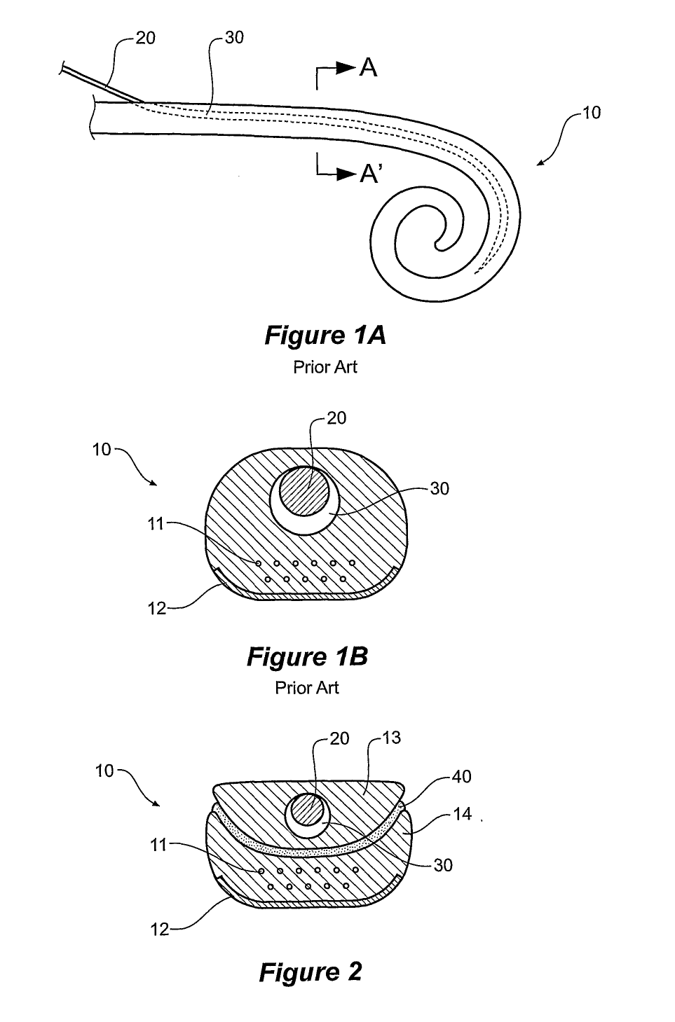

### DETAILED DESCRIPTION

The various aspects of the present invention will now be described in detail with reference to one or more embodiments of the invention, examples of which are illustrated in the accompanying drawings. The examples and embodiments are provided by way of explanation only and are not to be taken as limiting to the scope of the invention. Furthermore, features illustrated or described as part of one embodiment may be used with one or more other embodiments to provide a further new combination.

It will be understood that the present invention will cover these variations and embodiments as well as variations and modifications that would be understood by the person skilled in the art.

During the following description, the term “lumen” will be used to refer to the cavity formed within the electrode lead or a portion of the electrode lead for receiving an insert such as a stylet.

While the various aspects of the present invention will be described with specific reference to a cochlear implant, it will be understood that the principles of the various aspects of the present invention may be applied to other types of medical implants. For example:

ABI (Auditory Brainstem Implant, electrode for hearing, placed in the brainstem) such as Cochlear Corporation's Nucleus 24 [R] Multichannel Auditory Brainstem Implant (Multichannel ABI)

The auditory brainstem implant consists of a small electrode that is applied to the brainstem where it stimulates acoustic nerves by means of electrical signals. The stimulating electrical signals are provided by a signal processor processing input sounds from a microphone located externally to the user. This allows the user to hear a certain degree of sound.

FES (Functional Electrical Stimulation) is a technique that uses electrical currents to activate muscles and/or nerves, restoring function in people with paralysis-related disabilities.

Injuries to the spinal cord interfere with electrical signals between the brain and the muscles, which can result in paralysis.

This SCS (Spinal Cord Stimulator) system delivers pulses of electrical energy via an electrode in the spinal area and may be used for pain management. An example of a commercially available system is the RESTOREPRIME system by Medtronic, Inc, USA.

FIG. 2 shows a cross-sectional view of an electrode lead 10 according to one embodiment of the present invention. Shown is the electrode lead 10 provided as two separate parts—a lumen portion 13 and a main electrode lead portion 14. Lumen portion 13 defines the lumen 30, for receiving stylet 20, while main electrode lead portion 14 contains the plurality of electrode contacts 12 and respective conductive pathways or electrode conductive wires 11, which convey stimulation signals to respective electrode contacts 12 for stimulation of the patient's or recipient's tissue.

Connecting lumen portion 13 to main electrode lead portion 14 is a temporary connector 40. Temporary connector 40 may be a polymer, and in one example, a resorbable polymer, such as Polyacrylic acid (PAA), Polyvinyl alcohol (PVA), Polyactic acid (PLA) or Polyglycolic acid (PGA), (or any combination thereof) which, after a preset time lapse, or upon contact with fluid, begins to soften or dissolve, allowing lumen portion 13 to be separated from main electrode lead portion 14. After implantation of the implant into the patient's cochlea, the surgeon can pull out both the stylet 20 and the lumen portion 13, leaving only the main electrode lead portion 14 in the cochlea. The result is an implant of reduced bulk, and no easy path for passage of fluid to cause infection.

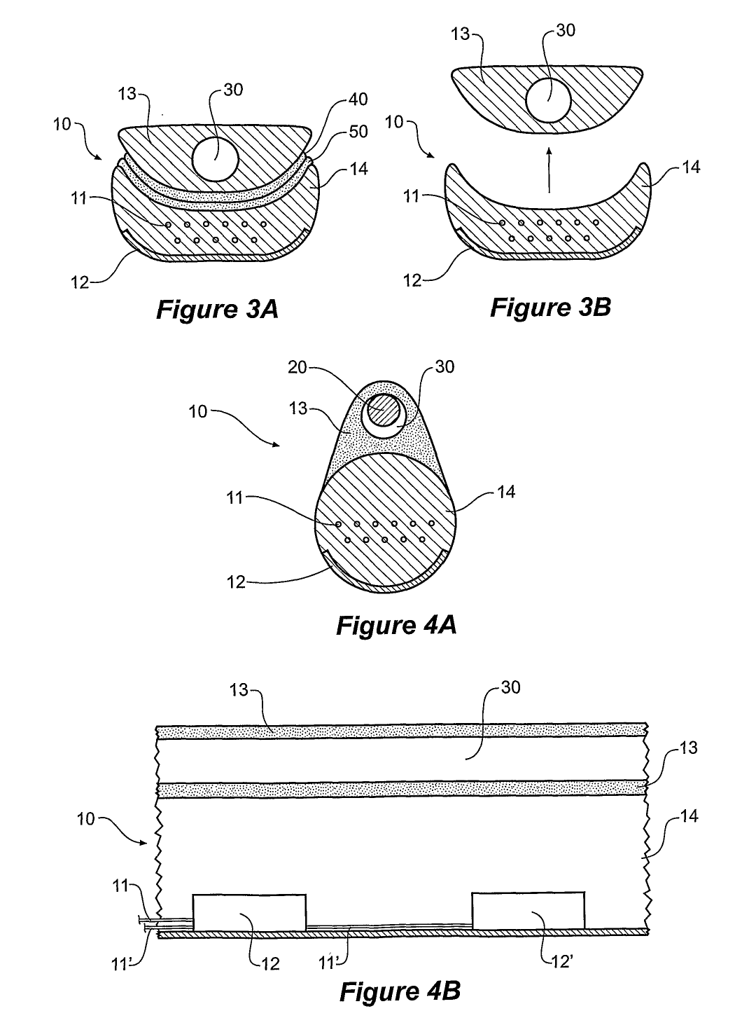

FIG. 3A shows a variation of the electrode lead 10 of FIG. 2. Specifically, electrode lead 10 is still made up of the lumen portion 13 and the main electrode lead portion 14 with wires 11 and electrode contact 12, however, instead of only a single temporary connector 40, there is now a second temporary connector 50.

In this embodiment, temporary connector 40 may be a similar material to that used in the examples given with reference to FIG. 2, such that it will soften or dissolve after a preset time period and/or contact with fluid. This will be designed to allow relatively fast separation of lumen portion 13 from main electrode lead portion 14 to allow removal of lumen portion 13 as previously described.

FIG. 3B shows the lumen portion 13 with lumen 30 separated from main electrode lead portion 14 with both temporary and second temporary connectors dissolved.

Second temporary connector 50 may be of a different material such as a resorbable polymer—for example Polyacrylic acid (PAA), Polyvinyl alcohol (PVA), Polyactic acid (PLA) or Polyglycolic acid (PGA). This material is designed to dissolve more slowly than temporary connector 40, and may slowly release drugs such as antibiotics or neurotrophins into the cochlea to facilitate the healing and recovery process and reduce the likelihood of infection.

In a further embodiment of this aspect of the invention, the lumen portion 13 defining lumen 30 may be made entirely of the dissolving material as previously mentioned. FIG. 4A shows electrode lead 10 made up of lumen portion 13 defining lumen 30, and main electrode lead portion 14 containing wires 11 and electrode contact 12. In this embodiment, lumen portion 13 is made substantially of the dissolving material and is disposed on top of main electrode lead portion 14. Stylet 20 will in use, be received in lumen 30 during or prior to the implant procedure, and after a preset amount of time or contact with fluid, the entire lumen portion 13 will begin to dissolve, leaving stylet 20 completely separated from main electrode lead portion 14, and able to be pulled out of the cochlea by the surgeon. Alternatively, the surgeon may remove the stylet during insertion, leaving the lumen portion 13 to dissolve over time. In this embodiment, the lumen portion 13 may also release drugs while dissolving.

FIG. 4B shows a side view of an electrode lead 10 according to this embodiment as shown in FIG. 4A. In this view, stylet 20 is not shown. Shown is main electrode lead portion 14 supporting electrode contacts 12 and 12′ with respective electrode wires or conductors 11, 11′. Lumen portion 13 is shown, connected to main electrode portion 14, and defines lumen 30. After insertion, lumen portion 13 slowly dissolves, removing lumen 30 and leaving behind main electrode lead portion 14 with associated electrode array.

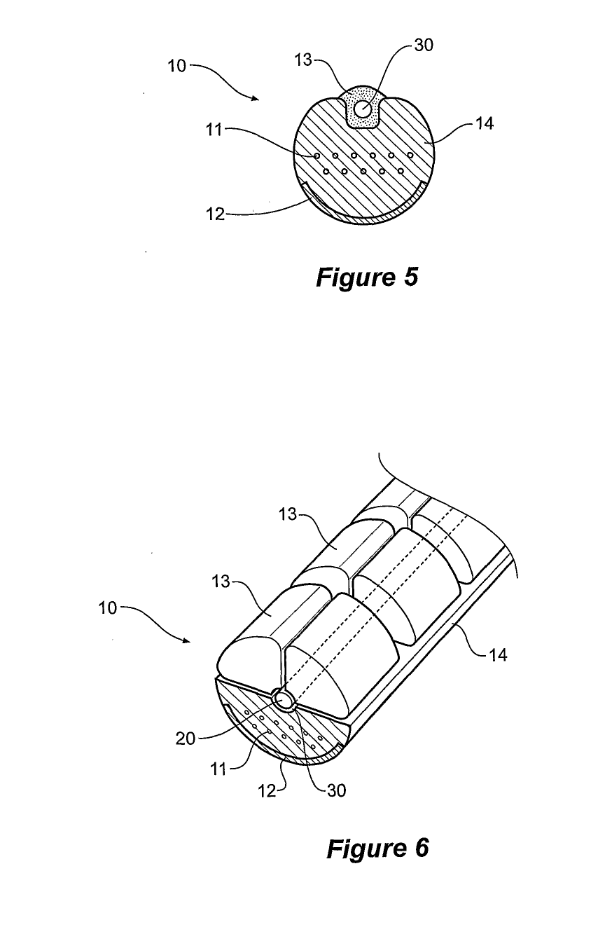

In yet a further embodiment as shown in FIG. 5, main electrode lead portion 14 may be shaped to provide a channel in which at least a part of lumen portion 13 may be disposed. As in the embodiment described above in relation to FIGS. 4A and 4B, lumen portion 13 is made up entirely of the dissolving material, which upon dissolving releases stylet 20 (not shown) from main electrode lead portion 14 allowing it to be removed. This particular design provides for an even less bulky main electrode lead portion that is left in the patient's cochlea.

FIG. 6 shows a similar design as that shown in FIG. 5; however, main electrode lead portion 14 defines a lower half of electrode lead 10, while lumen portion 13 defines the upper half of electrode lead 10. In this embodiment, lumen 30 is defined by both portions, and upon dissolving of the lumen portion 13 material, stylet 20 is freed to be removed by the surgeon.

According to second and third aspects of the present invention, there is shown an embodiment of electrode lead 10 in which no stylet 20 and therefore no lumen 30 is required at all.

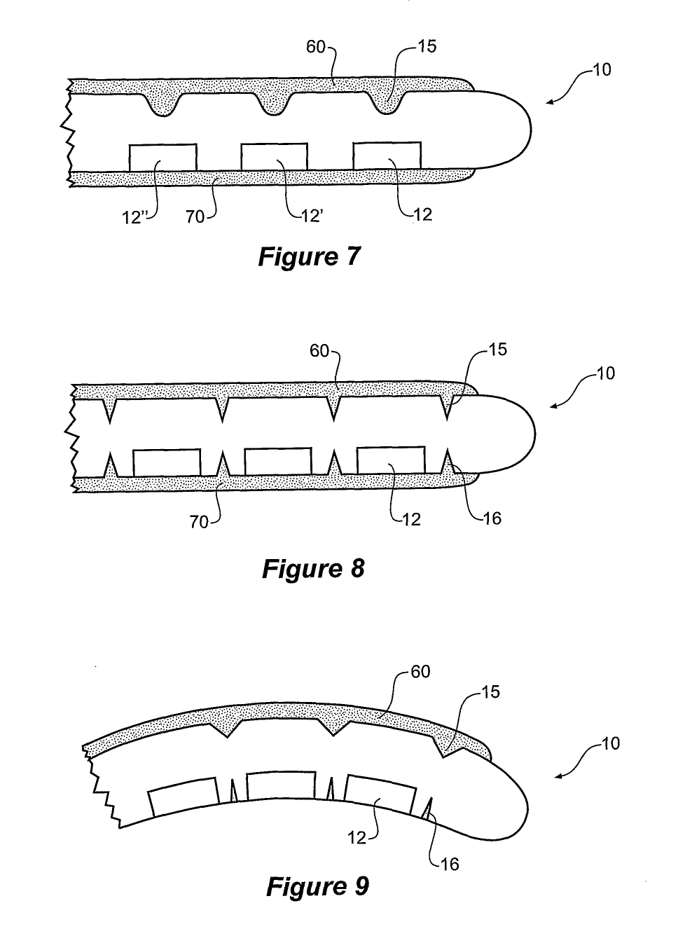

FIG. 7 shows electrode lead 10 having electrode contacts 12, 12′ and 12″ with a layer of swelling material 60 which upon contact with fluid such as saline or water, or by some other trigger such as humidity, begins to swell. This causes a positive pressure on the surface of electrode lead 10, causing it to bend or curl. This allows the electrode lead 10 to be manufactured without an inherent curl, providing a much more simplified manufacturing and handling process, and removes the need for a lumen and stylet.

The function of this aspect of the invention may be enhanced by designing electrode lead 10 to have one or more grooves 15 which are filled with the swelling material 60. This further enhances the curling action of electrode 10 as material 60 swells.

Examples of suitable materials for swelling material 60 include any type of suitable water-expanding material. One example is Silastic A™ silicone polymer, mixed for example with a finely-ground NaCl as will be known to the person skilled in the art. The curling effect can be even further enhanced by proving a layer of dissolvable material 70 on the side opposite the swelling material. Dissolving material 70 provides a counter force to swelling material 60 when it is present; however this counter force disappears once material 70 has dissolved. This allows more precise design of curling parameters.

FIG. 8 shows a further alternative of the embodiment shown in FIG. 7, in which electrode lead 10 also has slits or grooves 16 on the side opposite to slits or grooves 15, and filled with the dissolving material 70. Having material 70 in slits or grooves 16 provides a greater counter curling force to the curling force provided by swelling material 60; however, the presence of slits or grooves 16 when devoid of dissolving material 70 facilitates the curling caused by swelling material 60. Examples of suitable dissolving materials include those previously mentioned.

FIG. 9 shows electrode lead 10 in its curled position as slits or grooves 15 are made larger by swelling material 60, while slits or grooves 16 are made smaller by the disappearance of dissolving material 70 (not shown in this view).

The degree of curling of electrode 10 can also be controlled by controlling the shape and volume of slits or grooves 15 and 16 during the manufacturing process for example. These may be balanced by the strength or resilience of the electrode 10.

In these embodiments, the dissolving layers of material 70 may also contain antibiotics, neurotrophins or other drug compounds.

It will of course be understood that any other combination of the layers, materials and slits or grooves may be used and will not be limited to only those illustrated.

While the embodiments described with reference to FIGS. 7 to 9 may not require a lumen and stylet, it will be appreciated that such can be provided if required.

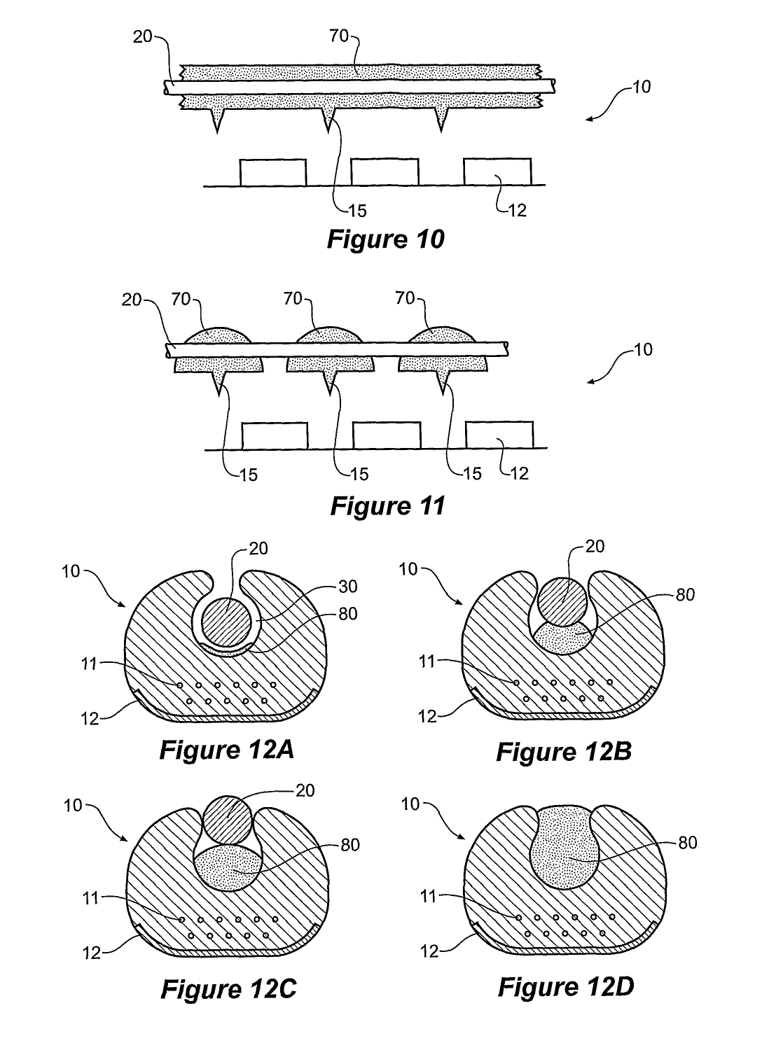

FIG. 10 shows a side cross-sectional view of an electrode lead 10 supporting electrode contacts 12 having slits or grooves 15 and dissolving material 70 in which stylet 20 may be embedded. Upon material 70 dissolving, stylet 20 is released to be removed from the cochlea, while also “emptying” slits or grooves 15 to allow them to “open” or expand, thereby causing electrode 10 to curl. In this embodiment, the dissolving material 70 may act to “pull together” the slits or grooves 15, and upon dissolving, release them to promote curving.

In another embodiment, the portion of lumen portion 13 above the lumen 30 as seen in FIGS. 10 and 11 may be made from a dissolving material while a lower portion (below lumen 30) may be made from a swelling material. Upon contact with fluid after insertion, the top portion dissolves, removing lumen 30, while the lower portion swells, to provide even greater curving forces as described above with reference to FIGS. 7-9.

FIG. 11 shows a similar embodiment to that of FIG. 10, except that dissolving material 70 is provided in discrete portions, retaining stylet 20 only at discrete points for release upon dissolving.

According to a fourth aspect of the present invention, electrode lead 10 may be shaped to define a partial lumen 30 or at least a portion of the lumen 30, to retain stylet 20 in place, and a swelling material may then be used to eject stylet 20 from electrode lead 10 as required after insertion. FIG. 12A shows electrode lead 10 shaped so as to provide a recess (which could be referred to as a partial lumen 30) for retaining stylet 20. Electrode lead 10 also supports wires 11 and electrode contacts 12.

Once electrode lead 10 is inside the cochlea of the patient, a swelling material 80 coating a lower portion of partial lumen 30 begins to swell as shown in FIG. 12B. This results in stylet 20 being urged out of partial lumen 30. FIG. 12C shows the swelling material 80 even more swollen, further expelling stylet 20 from partial lumen 30, and able to be removed by the surgeon. FIG. 12D shows the electrode lead 10 after the swelling material 80 has swelled to the extent that it entirely, or almost entirely, fills the cavity that was once lumen 30, thereby effectively removing lumen 30 from electrode lead 10. This embodiment will reduce the likelihood of fluids entering a cavity within electrode lead 10 that may subsequently promote infection.

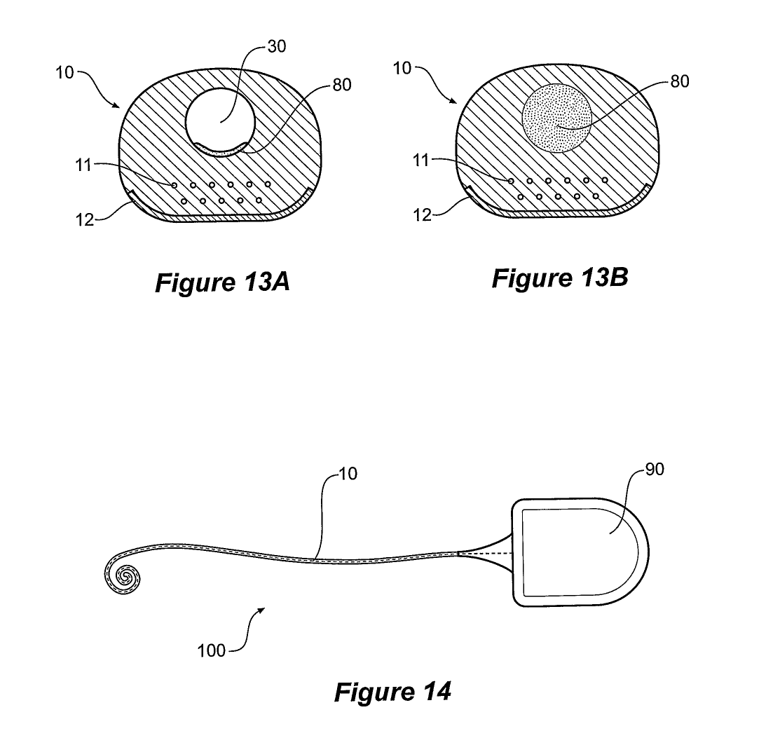

In a further variation of this embodiment, as shown in FIG. 13A, the electrode lead 10 can define the entire lumen 30, which has disposed therein, a material 80 that swells upon contact with fluid. In this arrangement, the stylet may be removed manually by the surgeon during or after the implantation, to leave lumen 30, which then is effectively removed, as material 80 begins to swell.

FIG. 13B shows electrode lead 10 without lumen 30, as material 80 has swelled to fill the cavity. Again, this embodiment will reduce the likelihood of fluids entering a cavity within electrode lead 10 that may subsequently promote infection.

FIG. 14 shows a cochlear implant 100 having stimulator 90 and electrode lead 10. Electrode lead 10 could have any one or more of the features described above.

The various forms of electrode lead 10 described above may be made in any manner known as would be apparent to the person skilled in the art. For example, in manufacturing the electrode lead 10 shown in FIG. 2, the following method may be used.

In order to form the electrode array, the electrode contacts 12, 12′, 12″ are placed in a U-shaped holding die. In this case, the electrode contacts 12, 12′, 12″ may be welded or otherwise electrically connected to their respective electrode wires or conductors 11, 11′, 11″ in sequential order, starting from the most proximal electrode contact. Once all of the wires 11, 11′, 11″ have been connected to their respective electrode contacts 12, 12′, 12″, a droplet of adhesive 41, such as adhesive silicone, is placed in the trough of each electrode contact in order to secure the wires in place.

A production stylet (for example, a PTFE coated wire) is suspended or otherwise placed over the electrode array before filling each trough with more silicone. The production stylet is used to hold the electrode contacts in spaced relationship to each other and provide further support to the electrode array, and is later removed to form a lumen in the lead. The holding die is then placed in an oven to cure the silicone.

The formed electrode lead in one form, could then be split into two portions—the main lead portion 14 and the lumen portion 13, and then reconnected by applying an amount of material 40 between them and allowing this to set.

In an alternative method, prior to the step of introducing the production stylet, silicone may be poured into the trough to a level just above the electrode contacts and wires, allowed to cure, and then applying a layer of material 40 over the partially-constructed electrode lead. This partially-constructed electrode lead may then have the remainder of the silicone applied over the layer of material 40 using a production stylet to form the lumen, to form the 2-part electrode lead as shown in FIG. 2, with material 40 therebetween.

In a further alternative method, the two parts 14 and 13 may be formed separately and then combined using material 40 as described above.

In forming the arrangement shown in FIG. 4A, the main lead portion 14 could be formed by conventional means, and then lumen portion 13 could be either formed separately and adhered to portion 14, or by pouring and allowing to set on top of portion 14, material 40, using a production stylet to form the lumen.

In forming the electrode lead as shown in FIG. 12A, the silicone could be poured up to about halfway up to the top of the production stylet. The production stylet could then be removed, and an amount of material 80 could be placed in the semi-lumen thus created, and then cured. The production stylet could then be reintroduced and the silicone continued to be poured to cover the production stylet to form the full lumen, and then processed as described above.

Once formed, the electrode lead 10 may then be removed from the U-shaped holding die and placed in a curved moulding die, if a curved electrode lead is desired, as will be known to the person skilled in the art. These methods may also be combined with appropriate parts or appropriately modified parts of methods as described in International Patent Application No. PCT/AU99/00391 (WO 00/71063) to the present applicant, previously incorporated by reference.

It will be understood that the above has been described with reference to particular embodiments and that many variations and modifications may be made within the scopes of the different aspects of the present invention.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

1. An electrode lead for a medical implant, comprising:
a lumen portion at least partially defining a lumen for receiving a stylet; and a main portion configured to carry one or more electrodes, wherein the main portion is removably connected to the lumen portion.

2. The electrode lead of claim 1, wherein the lumen portion and the main portion are elongate portions of an elongate electrode lead.

3. The electrode lead of claim 2, wherein the lumen portion and the main portion are releasably connected by a temporary connector comprising a degradable material.

4. The electrode lead of claim 3, wherein the degradable material is a resorbable polymer.

5. The electrode lead of claim 4, wherein the resorbable polymer is any one or more of Polyacrylic acid (PAA), Polyvinyl alcohol (PVA), Polyactic acid (PLA) or Polyglycolic acid (PGA).

6. The electrode lead of claim 1, wherein the lumen portion is made from a degradable material.

7. The electrode lead of claim 6, wherein the degradable material is a resorbable polymer.

8. The electrode lead of claim 7, wherein the resorbable material is any one or more of Polyacrylic acid (PAA), Polyvinyl alcohol (PVA), Polyactic acid (PLA) or Polyglycolic acid (PGA).

9. The electrode lead of claim 2, wherein the lumen portion is also connected to the main portion by a second temporary connector.

10. The electrode lead of claim 9, wherein the second temporary connector is a resorbable polymer.

11. The electrode lead of claim 1, wherein the lumen portion is comprised of that expands upon contact with fluid to fill the lumen.

12. The electrode lead of claim 12, wherein the electrode lead has an opening above the lumen such that upon the material expanding, a stylet in the lumen is urged out of the lumen.

13. The electrode lead of claim 1, wherein the medical implant is a cochlear implant.

14. A cochlear implant comprising a stimulator and an electrode lead supporting an array of electrode contacts, the electrode lead comprising:
a lumen portion at least partially defining a lumen for receiving a stylet; and
a main portion configured to carry one or more electrodes, wherein the main portion is removably connected to the lumen portion.

15. The cochlear implant of claim 14, wherein the lumen portion and the main portion, are elongate portions of an elongate electrode lead.

16. The implant as claimed in claim 15, wherein the lumen portion and the main portion are releasably connected by a temporary connector comprising degradable material.

17. The cochlear implant of claim 16, wherein the degradable material is a resorbable polymer.

18. The cochlear implant of claim 17, wherein the resorbable polymer is any one or more of Polyacrylic acid (PAA), Polyvinyl alcohol (PVA), Polyactic acid (PLA) or Polyglycolic acid (PGA).

19. The cochlear implant of claim 14, wherein the lumen portion is made from a degradable material.

20. The cochlear implant of claim 19, wherein the degradable material is a resorbable polymer.

21. The cochlear implant of claim 20, wherein the resorbable material is any one or more of Polyacrylic acid (PAA), Polyvinyl alcohol (PVA), Polyactic acid (PLA) or Polyglycolic acid (PGA).

22. The cochlear implant of claim 15, wherein the lumen portion is also connected to the main portion by a second temporary connector.

23. The cochlear implant of claim 22, wherein the second temporary connector is a resorbable polymer.

24-25. (canceled)
